(19) United States
(12) Patent Application Publication
(10) Pub. No.: US 2010/0100049 A1
(43) Pub. Date: Apr. 22, 2010

(54) SECUREMENT DEVICE FOR VASCULAR ACCESS SYSTEM

(76) Inventor: Mark W. Godfrey, Murrieta, CA (US)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 12/603,298

(22) Filed: Oct. 21, 2009

Related U.S. Application Data
(60) Provisional application No. 61/107,560, filed on Oct. 22, 2008.

(51) Int. Cl. A61M 25/02

(52) U.S. Cl. 604/180

(57) ABSTRACT

A securement device for a vascular access device, such as a catheter, includes a flexible base having a first side adapted for attachment to the patient and a second side. A support is coupled to the second side of the base and includes a surface that defines a recess configured to receive the vascular access device. The surface includes adhesive for securing the vascular access device within the recess and is oriented relative to the base for supporting an axis of the vascular access device at an angle relative to the patient. After the vascular access device has been secured in the recess by the adhesive, an occlusive dressing may be applied to cover the vascular access site.

### CROSS-REFERENCE TO RELATED APPLICATION

This application claims the benefit of and priority to U.S. Provisional Patent Application No. 61/107,560 filed Oct. 22, 2008, the entire contents of which are hereby incorporated by reference.

### FIELD OF THE INVENTION

The present invention relates to intravenous therapy, and more specifically to securement devices for supporting catheters and other vascular access devices.

### BACKGROUND

A large portion of modern health care treatments involve some form of infusion therapy. These therapies include the infusion of drugs, blood, and other fluids into the patient's body. Infusion therapy utilizes vascular access devices like catheters to provide this pathway. These access devices are preferably configured to be secured into place and protected from external agents and contaminates. The most common way to secure such devices in place is by taping the access device to the patient's skin. While this method is cost effective, studies have shown an increased rate of infection with this type of securement. Simple taping is also somewhat unstable and results in an inconsistent amount of support from patient to patient. The instability and inconsistency of taped devices present a risk of catheter dislodgements and increased discomfort for the patient. If the device becomes infected, is dislodged, or occludes, the IV must be reinserted and restarted to avoid health risks to the patient. This means additional cost to a healthcare facility in the form of nursing time, supply cost, and potentially extended patient hospitalization time, and increases the overall discomfort to the patient.

### SUMMARY

In some aspects, the invention provides a securement device for supporting a vascular access device relative to a patient. The securement device includes a flexible base having a first side adapted for attachment to the patient and a second side. A support is coupled to the second side of the base and includes a surface that defines a recess configured to receive the vascular access device. The surface includes adhesive for securing the vascular access device within the recess and is oriented relative to the base for supporting an axis of the vascular access device at an angle relative to the patient.

In other aspects, the invention provides a securement device for supporting a vascular access device relative to a patient. The securement device includes a base having a first side adapted for attachment to the patient and a second side. A support is coupled to the second side of the base and includes a pair of substantially trapezoidal sidewalls and a concave surface that extends between the sidewalls. The concave surface defines a recess that is configured to receive the vascular access device. The surface includes adhesive for securing the vascular access device within the recess and is oriented relative to the base for supporting an axis of the vascular access device at an angle relative to the patient.

In still other aspects, the invention provides a securement device for supporting a vascular access device relative to a patient. The securement device includes a flexible base having a first side adapted for attachment to the patient and a second side. A support is coupled to the second side of the base and includes a first body portion and a second body portion that are divided by a channel and moveable relative to one another. The first and second body portions each include respective surface portions that cooperate to define a recess. The recess is configured to receive the vascular access device and includes adhesive for securing the vascular access device within the recess. The recess is oriented relative to the base for supporting an axis of the vascular access device at an angle relative to the patient.

### BRIEF DESCRIPTION OF THE DRAWINGS

FIG. 1 is a top view of a catheter securement assembly attached to a patient.

FIG. 2 is an exploded perspective view of the catheter securement assembly of FIG. 1.

FIG. 3 is a perspective view of a catheter assembly for use with the catheter securement assembly of FIG. 1.

FIG. 4 is a perspective view of an indwelling support of the catheter securement assembly of FIG. 1, the indwelling support adapted to support the catheter assembly of FIG. 3.

FIG. 5 is a top view of the catheter securement assembly of FIG. 1 with an occlusive dressing removed.

FIG. 6 is a side view of the catheter securement assembly that, like FIG. 5, has the occlusive dressing removed.

FIG. 7 is a perspective view of an alternative embodiment of the indwelling support of FIG. 4.

### DETAILED DESCRIPTION

It is to be understood that the invention is not limited in its application to the details of construction and the arrangements of the components set forth in the following description or embodiments, or illustrated in the drawings. The invention is capable of other embodiments and of being practiced or being carried out in various ways. Also, it is to be understood that the phraseology and terminology used herein is for the purpose of description and should not be regarded as limiting.

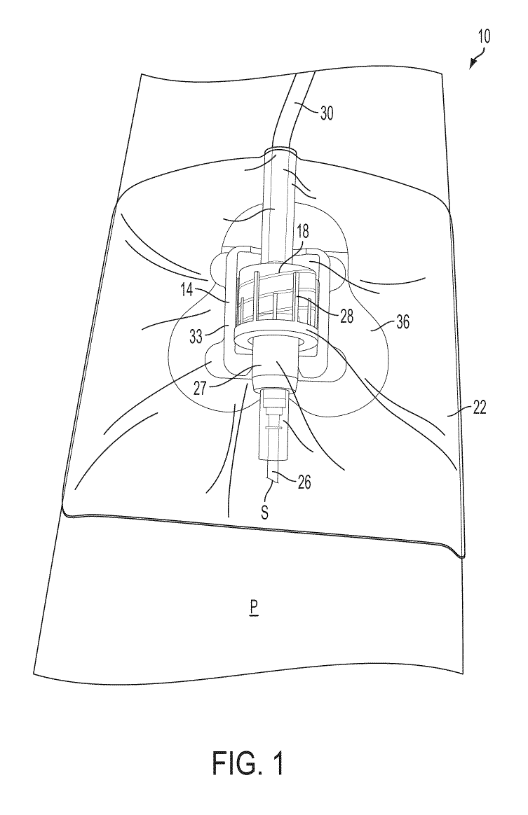

FIG. 1 illustrates a catheter securement assembly 10 associated with the administration of intravenous fluids into the vascular system of a patient P. The securement assembly 10 includes an indwelling support in the form of a cradle 14, a vascular access device (“VAD”) 18 (e.g., a catheter assembly, attached I.V. line, extension set hub, or various combinations thereof) supported by the indwelling support 14, and an occlusive dressing 22 configured to at least partially secure the indwelling support 14 and vascular access device 18 to the patient while also acting as a protective covering for the vascular access site. Although the securement assembly 10 is described below as being used specifically with a catheter assembly including a luer lock hub, the features and operational concepts associated with the securement assembly 10 may also be utilized with or include other types of vascular access devices.

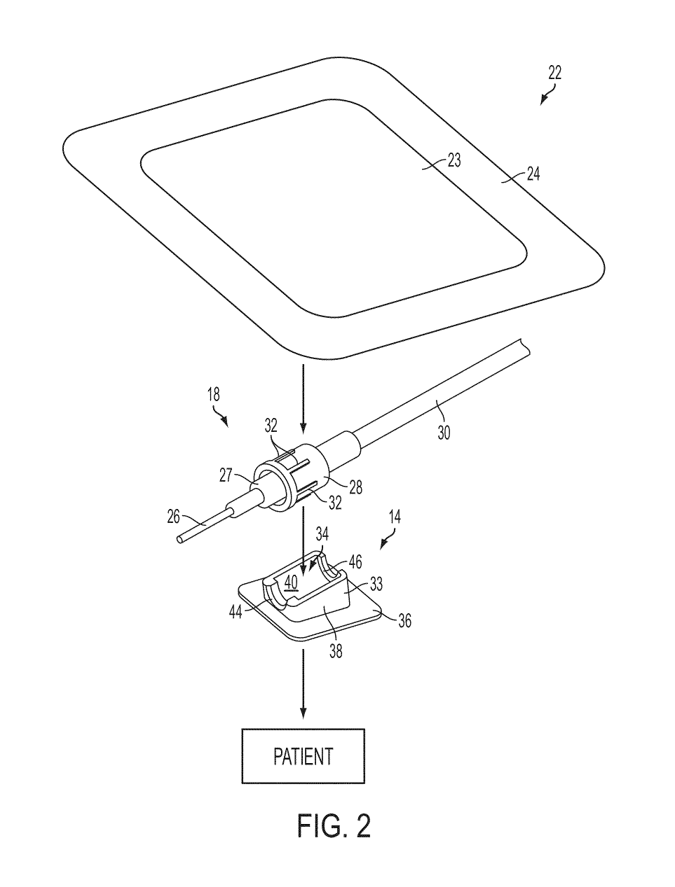

With reference to FIG. 2, the illustrated occlusive dressing 22 includes a central portion 23, and an outer portion 24. At least the outer portion 24 includes an adhesive for securing the occlusive dressing 22 to the patient and for providing additional support and stability to the indwelling support 14 and VAD 18. In some configurations the central portion 23 also includes an adhesive that may further secure the occlusive dressing 22 to the patient by adhering to and further stabilizing the VAD 18 and/or the indwelling support 14. The central portion 23 preferably is formed of a clear or translucent material to allow visualization of the puncture site and VAD 18. The central portion 23 is configured to be substantially impervious to bacteria (e.g., hydrogel) as generally known in the art, and may also include silver nitrate or similar antibacterial or therapeutic agents.

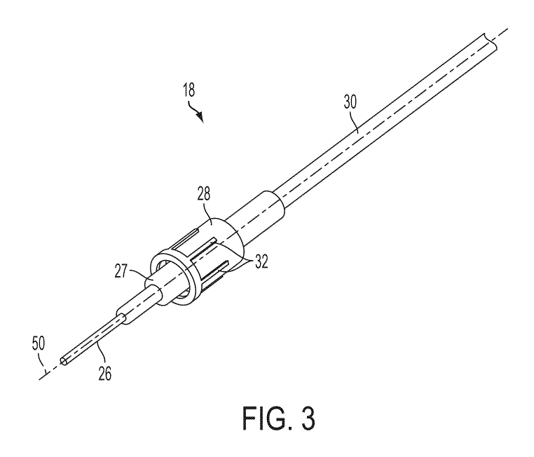

With reference also to FIG. 3 the VAD 18 in the illustrated construction is a catheter assembly that includes a catheter tube 26 joined to a catheter hub 27, a luer lock hub 28 removably connected to the catheter hub 27, and an extension tube 30 extending from the luer lock hub 28. Standing alone, the illustrated VAD 18 in the form of a catheter system generally is known in the art for conveying therapeutic liquids from the extension tube 30, through the junction formed by the catheter hub 27 and the luer lock hub 28, and into the patient via the catheter tube 26. The illustrated luer lock hub 28 is generally cylindrical and includes raised ridge portions 32 that facilitate gripping during attachment/detachment of the luer lock hub 28. Other configurations of luer lock hubs having other configurations of ridges, recesses, and gripping portions are also generally known in the art, all of which may be utilized in accordance with the teachings of the present invention.

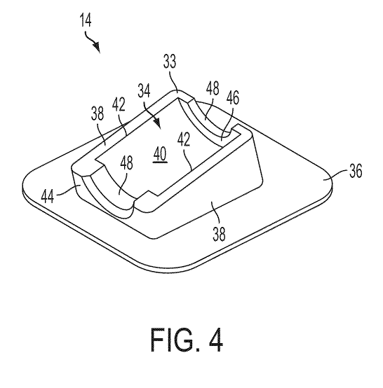

With reference also to FIG. 4, the indwelling support 14 is configured to support the VAD 18 and secure the VAD 18 to the patient P before the occlusive dressing 22 is applied over the indwelling support 14 and VAD 18. The indwelling support 14 includes a cradle or body portion 33 defining a recess 34 that receives and supports the VAD 18. The indwelling support 14 also includes a base portion 36 that is coupled to the body portion 33 and secured to the patient P. In the illustrated construction, the recess 34 is substantially semi-cylindrical to correspond to the above-described cylindrical luer lock hub 28 of the VAD 18. It should be appreciated that the indwelling support 14 can be modified for use with a variety of different VADs 18 by altering the shape and configuration of the recess 34 to correspond to the size and shape of the luer lock hub 28 or other component of the VAD in question. For example, the recess may include generally rectangular, triangular, conical or other geometries to provide appropriate support for the VAD 18.

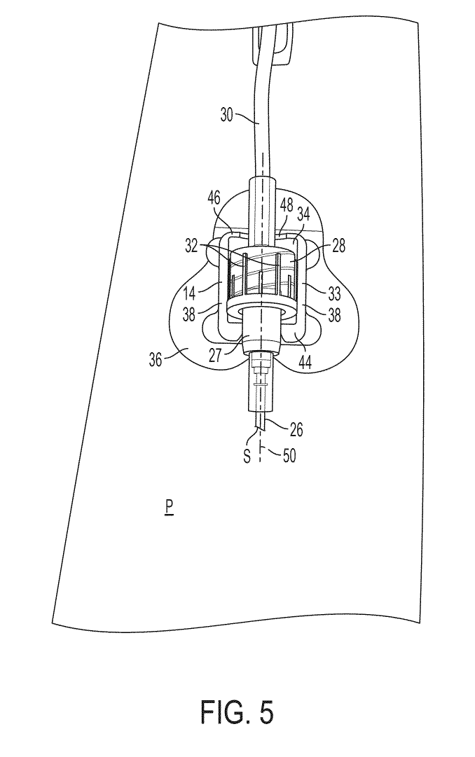

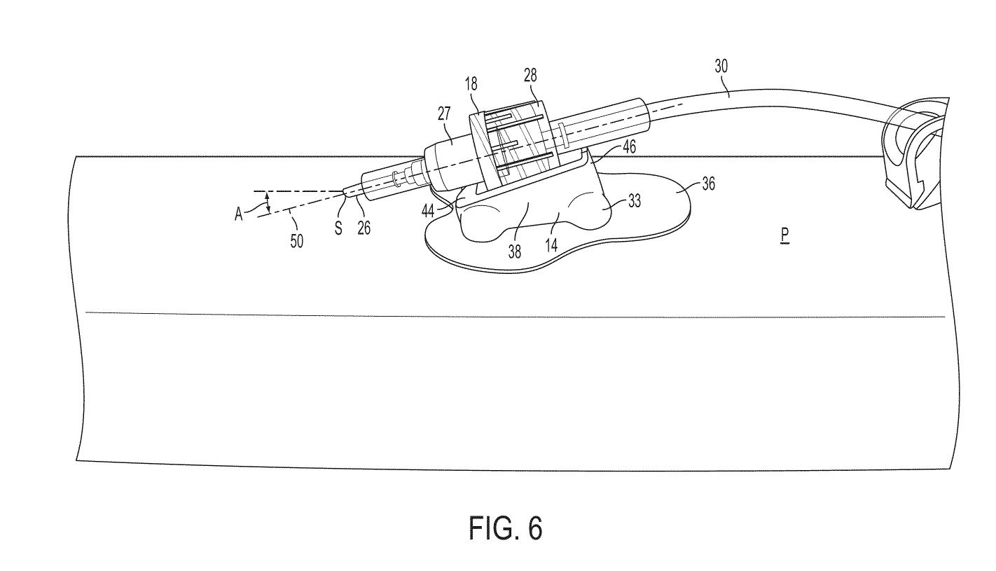

The body portion 33 includes a pair of opposed, generally trapezoidal sidewalls 38 that extend generally upwardly from the base portion 36. The recess 34 is defined by a generally cylindrical and concaved surface 40 that extends between upper edges 42 of the opposed sidewalls 38. A proximal end wall 44 extends between the sidewalls 38 at a location proximal to the patient's puncture site S (see FIGS. 5 and 6), and a distal end wall 46 extends between the sidewalls 38 at a location distal from the patient's puncture site S. The distal end wall 46 extends further from the base relative to the proximal end wall 44. The proximal and distal end walls 44, 46 each define a respective secondary recess 48. In the illustrated construction, the secondary recesses 48, like the recess 34, are generally cylindrical. The secondary recesses 48 each define a radius that is less than the radius of the recess 34. The reduced radii of the secondary recesses 48 are configured substantially to correspond to the diameters of the catheter hub 27 and the extension tube 30, which are less than the diameter of the luer lock hub 28.

The surface 40 of the recess 34 preferably includes an adhesive for securing the luer lock hub 28 to the body portion 33 when the luer lock hub 28 is inserted into the recess 34. The adhesive may cover substantially all of the surface 40 or may cover only selected portions or strips of the surface 40. The adhesive may be covered by a protective release layer that covers the adhesive and is removed prior to use. The surface 40 may include a resilient material to allow the adhesive to contact additional surface area of the luer lock hub 28. For example, in the illustrated embodiment in which the luer lock hub 28 includes ridge portions 32, the resilient material may allow the adhesive to contact the surfaces of the ridge portions 32 as well as the surfaces between the ridge portions 32, thereby enhancing securement of the VAD 18 to the indwelling support 14. In some embodiments, adhesive may also be provided on the exposed surfaces of the secondary recesses 48.

The base portion 36 is coupled to the body portion 33 and positioned between the body portion 33 and the patient's skin. The base portion 36 may be permanently or removably coupled to the body portion 33 by adhesives, hook and loop type fasteners, clips, rivets, or other mechanical means. The base portion 36 preferably is formed of a flexible thin material (e.g., fabric) and extends outwardly from the body portion 33. The material of the base portion 36 preferably is selected to allow circulation of moisture and air to the patient's skin. In the illustrated construction, the base portion 36 is formed of a hypoallergenic adhesive pad including hydrocolloid and adhesive material on an underside thereof that is non-irritating to the skin. A release layer covering the adhesive may be provided on the underside of the base portion 36.

With reference also to FIGS. 5 and 6, the indwelling support 14 is configured to support the VAD 18 at an angle relative to the patient P such that the catheter hub 27 is maintained in close proximity to the patient's puncture site S, thereby reducing the potential for relative movement between the patient and the catheter tube 26 that might otherwise lead to discomfort or dislodging of the catheter tube 26. More specifically, as can be seen when viewed from the side as in FIG. 6, the body portion 33 supports the VAD 18 at an angle A of between about 3 and about 5 degrees relative to the patient P (see also FIG. 6).

With continued reference to FIGS. 5 and 6, the VAD 18 defines an axis 50 extending generally through the luer lock hub 28 and the catheter hub 27. When the indwelling support 14 and VAD 18 are properly aligned and attached to the patient, the axis 50 extends through the patient's puncture site S. Because the surface 40 of the recess 34 is provided with adhesive, the indwelling support 14 can be configured such that no portion of the indwelling support 14 is positioned directly above any portion of the VAD 18, as can be seen in the top view of FIG. 5. Similarly, when viewed from the side as in FIG. 6, no portion of the indwelling support 14 extends above the axis 50. This configuration allows the VAD 18 to be moved downwardly and into the recess 34 generally without restriction or obstruction, where it is subsequently secured by the adhesive. This configuration substantially eliminates the need to snap, clip, strap, or otherwise secure the VAD 18 to the indwelling support 14 prior to application of the occlusive dressing 22 using structure that extends above or around the upper portion of the VAD 18, thereby simplifying the process of securing the indwelling support 14 and VAD 18 to the patient and reducing the likelihood of dislodging or excessively moving the VAD 18 during the same.

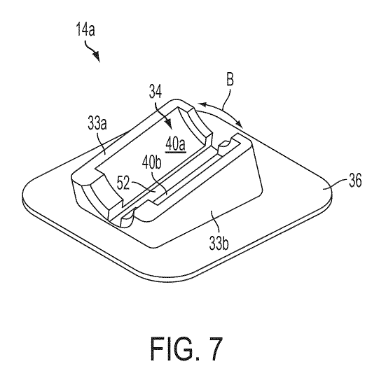

FIG. 7 illustrates an alternative embodiment of the indwelling support 14a. The embodiment of FIG. 7 is provided with a channel 52 that divides the indwelling support 14a in a longitudinal direction. The channel 52 substantially bifurcates the body portion into a first body portion 33a and a second body portion 33b that are substantial minor-images of one another. The body portion 33a includes a respective first surface 40a and the body portion 33b includes a respective second surface 40b. The first and second surfaces 40a, 40b together define the recess 34. The channel 52 extends in a direction that is substantially parallel to the axis 50 of the VAD 18 when the VAD 18 is positioned in the recess 34 (see FIG. 5). The channel 52 thereby allows the first and second body portions 33a, 33b to move generally inwardly and outwardly relative to one another in the direction of the arrow B in FIG. 7. In some embodiments, the channel 52 may be substantially T-shaped, including undercut portions that extend generally outwardly from a central portion of the channel 52 between the support surfaces 40a, 40b and the base 36.

By allowing movement of the first and second body portions 33a, 33b relative to one another, the indwelling support 14a of FIG. 7 can receive and retain a wider variety of VADs 18. For example, cylindrical luer lock hubs associated with pediatric devices (e.g., catheters, I.V. lines, extension sets, or combinations thereof) generally have a smaller diameter than cylindrical luer lock hubs associated with standard devices. When the indwelling support of FIG. 7 is used in combination with pediatric devices, the body portions 33a, 33b can be moved toward one another for engagement with the smaller pediatric luer lock hub. Similarly, if a larger luer lock hub is used, the body portions 33a, 33b can be moved away from one another to provide additional room for the larger luer lock hub. Movement of the body portions 33a, 33b relative to one another is at least partially facilitated by the flexible nature of the base portion 36.

In use, the puncture site S is prepared, the catheter tube 26 is inserted into the patient, and the luer lock hub 28 is connected to the catheter hub 27 in a known manner. If present, the release layer is removed to expose the adhesive within the recess 34 of the body portion 33 of the indwelling support 14. The luer lock hub 28 is then inserted into the recess 34 with the catheter hub 27 positioned in the secondary recess 48 of the proximal end wall 44 and the extension tube 30 positioned in the secondary recess 48 of the distal end wall 46. The luer lock hub 28 is then engaged with the adhesive lining the recess, thereby coupling the luer lock hub 28 to the body portion 33. If present, the release layer is removed from the underside of the base portion 36 and the base portion 36 is attached to the patient P adjacent the puncture site S. The adhesive on the occlusive dressing 22 is exposed, the occlusive dressing 22 is positioned over the indwelling support 14 and VAD 18, and the outer portion 24 of the occlusive dressing 22 is attached to the patient's skin. It should be appreciated that the outer portion 24 will also generally be attached to the extension tube 30, thereby also securing a portion of the extension tube 30 to the patient P.

What is claimed is:

1. A securement device for supporting a vascular access device relative to a patient, the vascular access device defining an axis, the securement device comprising:
a flexible base having a first side adapted for attachment to the patient and a second side; and
a support coupled to the second side of the base, the support including a surface that defines a recess configured to receive the vascular access device, the surface including adhesive for securing the vascular access device within the recess and oriented relative to the base for supporting the axis at an angle relative to the patient.

2. The securement device of claim 1, wherein the entire support is positioned between the axis and the patient.

3. The securement device of claim 1, wherein the vascular access device is secured within the recess solely by the adhesive.

4. The securement device of claim 1, wherein the base is formed of fabric.

5. The securement device of claim 1, wherein the surface is substantially semi-cylindrical and the recess opens away from the base.

6. The securement device of claim 5, wherein the semi-cylindrical surface engages the vascular access device over an arc of less than 180 degrees.

7. The catheter securement device of claim 1, wherein the support includes a pair of opposed and substantially trapezoidal sidewalls, and wherein the surface extends between the sidewalls.

8. The catheter securement device of claim 7, wherein the support includes a pair of end walls extending between the sidewalls, and wherein each end wall defines a respective secondary recess that is positioned adjacent the recess.

9. The catheter securement device of claim 8, wherein the pair of end walls includes a proximal end wall and a distal end wall, and wherein the distal end wall extends further from the base relative to the proximal end wall.

10. The catheter securement device of claim 7, wherein the recess is semi-cylindrical and defines a first radius, and wherein the secondary recesses are also semi-cylindrical, each secondary recess defining a radius that is smaller than the first radius.

11. The catheter securement device of claim 1, wherein the support defines a channel that divides the support into a first body portion and a second body portion that is moveable relative to the first body portion.

12. The catheter securement device of claim 11, wherein the first and second body portions are substantial mirror-images of one another.

13. The catheter securement device of claim 11, wherein the surface includes a first portion defined by the first body portion and a second portion defined by the second body portion.

14. A securement device for supporting a vascular access device relative to a patient, the vascular access device defining an axis, the securement device comprising:
a base having a first side adapted for attachment to the patient and a second side; and
a support coupled to the second side of the base, the support including a pair of substantially trapezoidal sidewalls and a concave surface that extends between the sidewalls and defines a recess, the recess configured to receive the vascular access device, the surface including adhesive for securing the vascular access device within the recess and oriented relative to the base for supporting the axis at an angle relative to the patient.

15. The securement device of claim 14, wherein the support includes a pair of end walls extending between the sidewalls, and wherein each end wall defines a respective secondary recess that is positioned adjacent the recess.

16. The securement device of claim 15, wherein the pair of end walls includes a proximal end wall and a distal end wall, and wherein the distal end wall extends further from the base relative to the proximal end wall.

17. The securement device of claim 15, wherein the recess is semi-cylindrical and defines a first radius, and wherein the secondary recesses are also semi-cylindrical, each secondary recess defining a radius that is smaller than the first radius.

18. The securement device of claim 14, wherein the entire support is positioned between the axis and the patient.

19. The securement device of claim 14, wherein the vascular access device is secured within the recess solely by the adhesive.

20. A securement device for supporting a vascular access device relative to a patient, the vascular access device defining an axis, the securement device comprising:
a flexible base having a first side adapted for attachment to the patient and a second side; and
a support coupled to the second side of the base, the support including a first body portion and a second body portion divided by a channel and moveable relative to one another, the first and second body portions each including respective surface portions that cooperate to define a recess configured to receive the vascular access device, the surface including adhesive for securing the vascular access device within the recess and oriented relative to the base for supporting the axis at an angle relative to the patient.

21. The securement device of claim 20, wherein the first and second body portions are moveable relative to one another in substantially inward and outward directions with respect to the axis.

22. The securement device of claim 20, wherein the first and second body portions are substantial mirror-images of one another.

23. The securement device of claim 20, wherein the entire support is positioned between the axis and the patient.

24. The securement device of claim 20, wherein the vascular access device is secured within the recess solely by the adhesive.
